(19) United States
(12) Patent Application Publication
(10) Pub. No.: US 2011/0146695 A1
(43) Pub. Date: Jun. 23, 2011

(54) Penile foreskin restraining device and method

(76) Inventor: Tony F. Taouil, Scottsdale, AZ (US)

(21) Appl. No.: 10/325,598

(22) Filed: Dec. 19, 2002

(51) Int. Cl. A61F 5/37

(52) U.S. Cl. 128/869; 128/883; 600/39; 600/41

(57) ABSTRACT

The penis foreskin restrictive device for an uncircumcised male wearer comprises a thin-walled sleeve of elastic material and has a diameter to firmly embrace the penis of the wearer when the penis is in the non-erection state. When applied, the sleeve is spaced from the glans penis so that during and after erection of the penis the foreskin is restricted against movement in a direction to cover the glans penis. A silk encased elastic belt is connected to one end of the sleeve and the body of the wearer to secure the sleeve against longitudinal movement in a direction toward the glan penis during and after erection.

This application is a divisional of application 60/335,233 filled on Nov. 30, 2001.

Nov. 30, 2001

### BACKGROUND OF INVENTION

The foreskin of the penis, if not fully withdrawn from the glans penis during sexual intercourse, will cause the male to experience reduced sensitivity and therefore reduced sexual satisfaction. It is therefore an object of this invention to provide a device and method for effecting the retraction of the foreskin from the glans penis and maintaining such retraction during sexual intercourse

### TECHNICAL FIELD

This invention relates generally to devices which are intended to promote enhanced sexual intercourse or copulation and more particularly.

### DESCRIPTIONS OF THE EMBODIMENTS

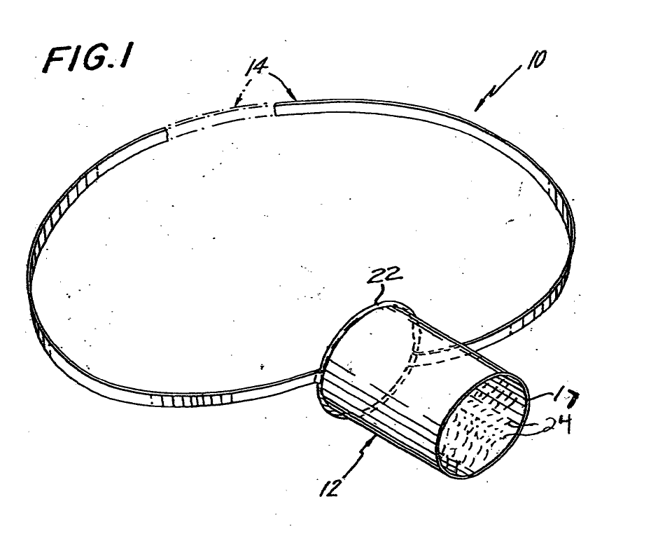

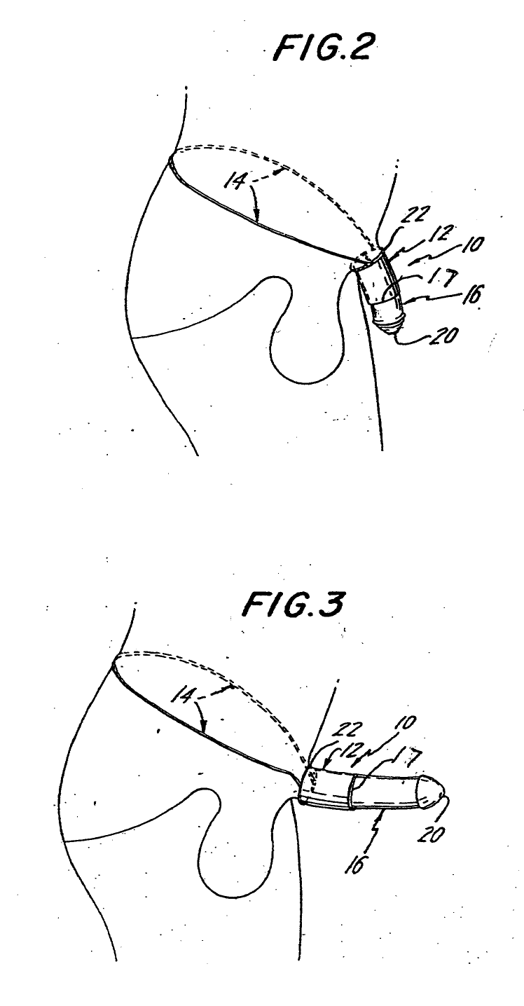

Referring now to FIG. 1, the foreskin restrictive device 10 is illustrated. The device 10 essentially comprises a thin-walled, open-ended hollow cylinder body portion or Sleeve 12. Sleeve 12 has a proximal portion which fits at the base of the penis, nearest to the body of the wearer, open ended 17, nearest to the glans penis. Sleeve 12 may will be made of elastic material such as, latex rubber. A belt made of silk encased with elastic 14 holds the device 10 in position on the wearer's penis 16 as best seen in FIGS. 2 and 3.

FIG. 2 illustrates that sleeve 12 is dimensioned in length so that the front end or portion 17 is spaced away from and does not touch nor cover the glans 20 of penis 16 when the penis is in the relaxed or non-errection state. The sleeve 12 is structured and arranged with a diametric dimension such that sleeve 12, when applied to penis 16 firmly embraces (in a manner similar to a condom) the penis. Sleeve 12 fits against the skin of the penis. In this way, during the erection of penis 16 the penis is gripped by the sleeve with increasing force thereby restricting the skin of the penis. This gripping restrains the foreskin against stretching or longitudinal movement and facilitates the extension of the glans 20 of penis 16 through the foreskin.

To further restrict the penis 16 from longitudinal movement while in a state of erection, a plurality of rearwardly directed cilia 24 can be incorporated into the interior of sleeve 12. The cilia may engage the penis when erect yet allow removal of the device when flaccid.

Belt 14 maybe, as shown in FIGS. 2 and 3, in the form of an elastic strip at opposite ends to the end portion 22 of sleeve 12. The end portion 22 of sleeve 12 may be peripherally reinforced in any suitable manner, as for example by being tightly rolled on itself or around an elastic ring (not shown), and to which belt 14 is connected. Alternately, belt 14 could be two ribbons (not shown) the ends of which would be connected together by tying or by having interlocking hook and loop connections, snaps or any other connecting mechanism on the front open end portion of the ribbons.

To use the device 10, the wearer, if securing means 14 is a continuous Elastic satin, steps within the elastic strip and brings device 10 up around his hips. Therefore, he places his penis 16 through sleeve 12 from open end 22 through the Front open end 17. The sleeve 12 is positioned on penis 16 close to the wearer's body or the base of penis 16. The positioning of device 10 is preferably accomplished while the penis 16 is in the relaxed or non-erection state so that during erection, sleeve 12 grips penis 16 with increasing force thereby restraining the penis skin and hence the foreskin from moving longitudinally and allowing glans penis to protrude through the foreskin. If device 10 is applied to the wearer while the penis is in the state of erection, the foreskin must be pulled back to expose the glans penis with the penis skin disposed under sleeve 12 to thereby embraced-by sleeve 12 and to hold the foreskin in the retracted condition. The securing means 14 prevents longitudinal movement of sleeve 12 during and after erection so that the foreskin is restrictec from recovering the glans penis before and after copulation or sexual intercourse. With the glans penis uncovered, the uncircumcised wearer enjoys heightened sensitivity and experienced improved sexual satisfaction It is contemplated that device 10 may be made in various sizes to accommodate the physical differenced in the uncircumcised users.

While the invention has been particularly shown and described with reference to embodiments thereof, it will be understood by those skilled in the art that various changes in form and details may be made therein without departing from the spirit and scope of invention.

1. A foreskin restrictive device comprising:
a sleeve having a proximal end and a open end
a belt connected to the proximal end of the sleeve:
wherein the sleeve is structured and arranged to fit snugly against skin of a penis of a wearer when the penis is in a non-erect state;
wherein the sleeve is structured and arranged to allow a glans penis to protrude beyond the open end of the sleeve;
wherein the belt connects around hips of the wearer to restrict the sleeve and the skin of the penis from moving longitudinally toward the glans penis during and after erection.

2. The device wherein said sleeve is made of latex

3. The device wherein said belt is an silk encased elastic of sufficient elasticity to encompass the hips of the wearer.

4. The device wherein the proximal end of the sleeve is reinforced.

5. The device wherein said belt is a silk encased elastic secured to the reinforced proximal end portion of the sleeve.

6. The device further comprising a plurality of cilia mounted within the sleeve.

7. A method for using a foreskin restrictive device comprising the following steps:
applying a sleeve to the wearer's belt.
wherein said sleeve is connected to a belt.
securing the belt around a wearer's waist
wherein during erection of the penis with increasing force thereby restricting the foreskin of the penis from moving longitudinally toward a glans penis; and
wherein wearing the sleeve allows the glans penis to protrude through the foreskin
